(19) United States
(12) Patent Application Publication
(10) Pub. No.: US 2007/0266957 A1
(43) Pub. Date: Nov. 22, 2007

(54) Antipathogenic Domestic Livestock House, Disinfectants for Domestic Livestock House, Disinfectants for Living Organisms, Feedstuffs and Drinking Water for Animals

(76) Inventors: Eisaku Tozaka, Kyoto (JP); Kazuo Takahashi, Osaka (JP)

Correspondence Address: MCGINN INTELLECTUAL PROPERTY LAW GROUP, PLLC, 8321 OLD COURTHOUSE ROAD, SUITE 200, VIENNA, VA, 22182-3817, US

(21) Appl. No.: 11/663,722

(22) PCT Filed: Aug. 25, 2005
(86) PCT No.: PCT/JP05/15455

§ 371 Date: Mar. 26, 2007

(30) Foreign Application Priority Data
Sep. 28, 2004 (JP) 2004-282571

(51) Int. Cl. A61K 36/13; A01K 31/18

(52) U.S. Cl. 119/437; 424/725; 424/770

(57) ABSTRACT

An antipathogenic domestic livestock house includes a building structure (chicken house) A1 for breeding or living a living organism B1, extracts of vegetable organisms of cupressaceae for preventing and eliminating pathogenic infections of the living organism B1, and a spreading means 1 for spreading the extracts of vegetable organisms of cupressaceae into an atmosphere surrounding the living organism B1.

### BACKGROUND OF THE INVENTION

1. Field of the Invention

The present invention directs to prevention and elimination against infection of living organisms due to pathogenic organisms including influenza viruses.

2. Description of the Related Art

Many people are infected by influenza viruses to become ill, and cause deaths every year. Although human beings are infected by human influenza viruses, new types of human influenza viruses appear year after year, and it results in a serious loss. It is considered that new type human influenza viruses are produced by variation or hybridization mating of avian influenza viruses with which birds such as chicken are infected, influenza viruses (for example, swine influenza viruses and the like) with which domestic livestock are infected, or existing human influenza viruses. In a related development, a serious problem arose from end of the year 2003 to 2004 wherein such a possibility that human being is directly infected by highly pathogenic avian influenza virus in an area extending from Southeast Asia to East Asia, North America and the like is pointed out. In also Japan, there arose death en masse of chickens, incineration of chickens and eggs bred in the same house in which dead chickens were bred, and a runup of prices in chicken meat and hen egg, resulting in substantial human and economical losses with respect to not only chicken raisers, but also the public at large (see a book entitled “TORI-INFURUENNZA NO KYOUI—HONTOU NO KOWASA HA KOREKARADA! (Threat caused by avian influenza—True anxieties will begin from now on!)” . Harue Okada, publishedby Kawade Shoboh Shin-sha, May 20, 2004).

As countermeasures against influenza viruses, an administration of a vaccine suitable for each virus or a medicine such as neuraminidase inhibitor is considered to be effective. In these circumstances, a vaccine for an influenza virus which is prospected to occur in epidemics in that year has been developed annually. However, an influenza virus occurs in epidemics in reality is not the one which has been previously prospected. In the first place, such medicine itself is expensive, and there is a problem in that it comes short of a vaccine particularly in case of reaching epidemic proportions. Furthermore, since a development for human vaccine has a priority, a development for bird vaccine is left until later, so that it might bring about a problem of damage in death en masse of birds. Moreover, the above-mentioned damage expands over the borders all over the world for a short period of time in such remarkable progress in means of transportation in these days. Thus, delay in taking action brings about great deal of global havoc.

At the same time, there is such finding that extracted oils of vegetable organisms of cupressaceae (called by the names of hinoki cypress oil, cypress oil and the like) such as Aomori cypress, Taiwan cypress and the like exhibit antibacterial activities. In this connection, such a technique as to an apparatus wherein a drug solution accumulated in a container is sprayed on air in doors which has been sucked in, and then, the air so sprayed is exhausted, whereby suspended particulate matters such.as microorganisms, viruses, and pollens, house dusts, harmful gases and the like in the air in doors are eliminated, and in this case hinoki cypress oil and the like are used as the drug solution is proposed (for example, see Japanese Patent Application Laid-Open No. 2000-210521).

The former patent document discloses that when a tea seed oil (tea extraction oil containing catechin) is used as a medical substance, it is effective for influenza virus, while when hinoki cypress oil is used as a medical substance, it is effective for MRSA (mesitylene resistant Staphylococcus aureus) or Escherichia coliform bacillus, and grounds in such difference are in that spectra for sterilization/prevention and elimination differ dependent on types of vegetable essential oils. However, specific reported test results are in that an aqueous solution containing hinoki cypress oil exhibits low toxicity with respect to an animal (mouse), that insects including mites as the major component exist in a drug solution (an aqueous solution containing hinoki cypress oil), while cystoid spores of fungus disappear after two weeks as a result of operation tests of the above-described apparatus. Accordingly, it is not necessarily clear from the former patent document whether or not hinoki cypress oil has a resistance to MRSA or a resistance to Escherichia coli activity, and whether or not there is anti-influenza virus activity, when a medical substance is tea seed oil.

On the other hand, the present inventor has studied and developed insecticides for termite control and the like with taking atmosphere and health into consideration by using cypress oil being a natural product as a result of paying attention to preventing and eliminating actions upon termites due to extracted oils (cypress oil) of vegetable organisms of cupressaceae contained therein, and in the process thereof the present inventor has newly found that a possibility of performing antiviral action, above all things, resistance to actions of influenza virus including avian virus by extracted oils of vegetable organisms of cupressaceae.

Accordingly, a principal object of the present invention is to provide an antipathogenic domestic livestock house wherein extracted oils of vegetable organisms of cupressaceae which can effectively prevent viral infections or the other pathogenic organism infections with respect to animals (including birds) and human being are used.

### SUMMARY OF THE INVENTION

According to the present invention, an antipathogenic domestic livestock house may comprise a building structure for breeding or living a living organism; extracts of vegetable organisms of cupressaceae for preventing and eliminating pathogenic infections of the living organism; and a spreading means for spreading the extracts of vegetable organisms of cupressaceae into an atmosphere surrounding the living organism.

Further, according to the present invention, an antipathogenic domestic livestock house may comprise a building structure for breeding or living a living organism; a liquid containing extracts of vegetable organisms of cupressaceae for preventing and eliminating pathogenic infections of the living organism; and a sparging means for sparging the liquid on at least a ceiling, a wall, or a floor in the building structure.

According to the invention, a disinfectant for a domestic livestock house may comprise extracts of vegetable organisms of cupressaceae served for disinfecting the domestic livestock house for breeding or living a living organism.

According to the invention, a disinfectant for living organisms may comprise extracts of vegetable organisms of cupressaceae served for suppressing pathogenic infections of the living organisms.

According to the invention, a feedstuff may comprise extracts of vegetable organisms of cupressaceae served for suppressing pathogenic infections of the living organisms.

According to the invention, a drinking water for animals may comprise extracts of vegetable organisms of cupressaceae served for suppressing pathogenic infections of the animals such as domestic fowls, and domestic animals. In this case, the animals include those which can be bred by human being other than domestic fowls and domestic animals, and do not include human being. More specifically, the drinking water for animals of the invention excludes beverages drunk principally by human being such as functional beverages.

Vegetable organisms of cupressaceae are evergreen needle-leaved trees distributed in the world. Typical examples of them include Aomori cypress (Hinokiasunaro, Asunaro genus), Taiwan cypress, Hinoki, Alaska cedar (Hinoki genus), Italian cypress (Cypress genus), Juniperus rigida (Juniperus rigida genus), Western redcedar or American arborvitae (Thuja standishii genus), and Incense-cedar (Calocedrus formosana genus). Extracted oils of vegetable organisms of cupressaceae (hereinafter referred optionally to as “cypress oil”) are obtained in a condition wherein they are separated from extraction water through steam distillation of sawdust produced at the time of lumber sawing, a scrap wood or the like. The extracted oils contain acidic oil components (about 3 to 4% by weight) consisting essentially of phenols (hinokitiol, β-dolabrin, 1-rosinacid, carbachol, andthelike), and neutral oil components (about 96 to 97% by weight) consisting essentially of sesquiterpenes (thujopsene, p-cymene, dihydrocymene, cedrol, widdrol and the like). Some amount of the acidic oil components are contained in the extraction water. Accordingly, the extracts of vegetable organisms of cupressaceae used in the present invention may be either of the extracted oils and the extraction water, or the mixture thereof, or further specified components (including one or two or more of the mixture thereof) contained in the extracted oils or the extraction water.

When the extracts such as extracted oils of vegetable organisms of cupressaceae are spread or sparged by spreading or sparging means into the atmosphere inside a domestic livestock house, living organisms in the domestic livestock house inhale the extracts, so that the extracts adhere to internal mucosae of mouth, nose, throat and the like, whereby infections with a variety of viruses including influenza viruses, or the other pathogenic organisms can be very effectively prevented. As a matter of course, when the extracts are allowed to be in contact with living organisms in the form of a disinfectant for living organisms, or when the extracts are applied to living organisms in the form of a foodstuff, the same advantageous effects as mentioned above are achieved. Furthermore, when the extracts are applied to living organisms in the form of a drinking water for animals in the case where the living organisms are animals such as domestic fowls, and domestic animals, the quite same advantageous effects can be also achieved.

A preferred example of the spreading means includes the one for sparging or disposing a porous material with which a liquid containing the extracted oils or the extracts (hereinafter referred to as “extracted oils and the like”) is adsorbed on a floor, under the floor, or a ground of the domestic livestock house, and the one for atomizing the extracted oils and the like by means of an atomizer inside the domestic livestock house. As the porous material, a variety ofmaterials are applicable irrespective of they are inorganic materials, organic materials, natural or artificial materials. A specific example of the porous material includes wood pellets, zeolite agglomerate having a great number of fine pores, zeolite particles, ceramics particles, diatomaceous earth agglomerate, diatomaceous earth particles, organic high-molecular particles, and mixed particles of organic and inorganic materials. As the atomizer, a variety of devices are applicable irrespective of types of the devices, so far as a device has a function to accumulate liquid extracted oils and the like, and to spray the atomized extracted oils and the like to discharge the same into the air. An example of the liquid containing the extracted oils includes a solution prepared by diluting the extracted oils into a suitable solvent, a suspension prepared by dispersing the extracted oils into water, and an aqueous solution and the like prepared by mixing the extracted oils with water using a suitable emulsifier. In the case where porous particles with which the extracted oils and the like are adsorbed are sparged or disposed as an antipathogenic drug or an antiviral drug, the liquid containing the extracted oils and the like adsorbed into the fine pores of the porous particles volatile gradually to be diffused into the atmosphere, so that infection preventing effects can be maintained for a comparatively long period of time. On the other hand, when the extracted oils and the like are positively sprayed by means of an atomizer, infection preventing effects can be instantly obtained.

Any type of sparging means may be used so far as the sparging means can sparge the liquid as mentioned above to any of a roof (ceiling), walls, and floors of the domestic livestock house.

Moreover, the disinfectant for living organisms is preferably applied by sparging or disposing a porous material with which a liquid containing the extracted oils and the like is adsorbed to on a floor, under the floor, or a ground of the domestic livestock house wherein as the porous material, a variety of materials are applicable irrespective of they are inorganic materials, organic materials, natural or artificial materials. A specific example of the porous material includes wood pellets, zeolite agglomerate having a great number of fine pores, zeolite particles ceramics particles, diatomaceous earth agglomerate, diatomaceous earth particles, organic high-molecular particles, and mixed particles of organic and inorganic materials.

A specific example of the antipathogenic domestic livestock house includes buildings such as a domestic fowl house used for domestic fowls, domestic animal house used for animals (mammals other than human being), and an accommodation unit used for human being. The “domestic fowl house” in the invention may be any building for breeding birds, and thus breeding facilities in a chicken farm, birds breeding facilities or a bird house in a zoo and the like, and a bird house used in general homes and the like are included. In this case, classes of birds are not specified. The “domestic animal house” may be any building for. breeding animals excluding birds and human being, and thus, breeding facilities in a livestock farm, breeding facilities in a zoo and the like, a pet house, and a cage and the like used for pets and the like are included. In this case, classes of domestic animals (animals) are not particularly specified. The “building such as an accommodation unit” may be any type of a building in which peoples live and cope with life, or act, and thus general buildings, business buildings, condominium buildings, apartment buildings, public facilities, factories, research facilities and all of such buildings are included.

The present invention is effective for prevention of viral infection diseases, particularly avian influenza (bird pest) derived from avian influenza viruses (including highly pathogenic avian influenza viruses), animal influenza derived from animal influenza viruses (swine influenza viruses, equine influenza viruses and the like), and influenza derived from human influenza viruses (A, B, and C), and particularly the invention is effective for prevention of influenza derived from H3 type and H5 type influenza viruses in H1 to H15 subtypes of A type influenza virus in the case when the antipathogenic domestic livestock house and the disinfectant for domestic livestock house according to the invention are applied to domestic fowl houses, domestic animal houses, accommodation units for human being use and the like, or the case when the disinfectant for living organisms and the feedstuff for living organisms according to the invention are applied to living organisms, in addition, the drinking water for animals according to the invention is applied to animals.

Furthermore, the present invention is effective for prevention of proliferation of salmonella in domestic fowl houses, domestic animal houses, accommodation units and the like, infections in living organisms with salmonella, contamination of animals and productions with salmonella in the case when the antipathogenic domestic livestock house and the disinfectant for domestic livestock house according to the invention are applied to domestic fowl houses, domestic animal houses, accommodation units for human being use and the like, or the case when the disinfectant for living organisms and the feedstuff for living organisms according to the invention are applied to living organisms, in addition, the drinking water for animals according to the invention is applied to animals.

Moreover, specific examples as to relationships between pathogenic organisms or the like and diseases which have a possibility of preventing effectively infections therewith are as follows.

(i) In case of applying the invention to a domestic fowl house (principally, a chicken house):

RNA virus: avian reovirus disease, infectious bursal disease, infectious bronchitis, Newcastle disease, avian leukoses, avian encephalomyelitis, and swollen head syndrome.

DNA virus: fowl pox, infectious laryngotracheitis, Marek's disease, egg drop syndrome, and avian anemic viral infection.

Mycoplasma: avian mycoplasma infection.

Gram-negative aerobic bacillus: pseudomonas aeruginosa infection.

Gram-negative facultative anaerobic bacillus: coli-bacillosis, salmonellosis, pullorum disease, pasteurellosis, fowl cholera, and infectious coryza.

Gram-positive coccus: staphylococcal disease, and streptococcosis.

Gram-positive spore forming bacteria: necrotic enteritis, clostridial infection disease, and botulism.

Fungus: mycosis.

Protozoa: coccidiosis, leucocytozoonosis, cryptosporidiosis, and histomoniasis.

Eelworm: hairworm disease, heterakis gallinarum disease, and ascaridia galli disease.

Cestoid: teniasis.

Arthropod: dermanyssus gallinae disease, ornithonyssus sylviarum parasitism, fowl mange, and fowl phthiriasis.

(ii) In case of applying the invention toa domestic animal house (pig house):

RNA virus: rotavirus disease, hog cholera, swine getah virus disease, Japanese encephalitis, transmissible gastroenteritis of swine, contagious encephalomyelitis, swine leukemia, enterovirus encephalomyelitis, porcine reproductive and respiratory syndrome, and porcine epidemic diarrhea.

DNA virus: swinepox, Aujeszky's disease, porcine cytomegalovirus disease, and porcine parvovirus infection disease.

Rickettsia: eperythrozoonosis.

Mycoplasma: swine enzootic pneumonia, mycoplasma infection disease.

Spirochaeta: swine dysentery, and leptospirosis.

Spirillum: proliferative haemorrhagic enteropathy, porcine intestinal adenomatosis, and campylobacteriosis.

Gram-negative aerobic bacillus: atrophic pyelonephritis, and pseudomonas aeruginosa disease.

Gram-negative facultative anaerobic bacillus: colibacillosis, cerebrospinal blood circulatory system disease, premature colon disease, swine pullorum, edema disease, the other colibacillosis, salmonellosis, pasteurellosis, klebsiella disease, actinobacillus disease complication, pleuropneumonia, and Haemophilus parasuis.

Gram-negative anaerobic asporogenic bacillus: fusobacterium disease.

Gram-positive coccus: staphylococcus disease, exudative dermatitis, and streptococcus disease.

Gram-positive spore-forming bacillus: necrotic enteritis, clostridial infection disease, tetanus, and malignant edema.

Gram-positive asporogenic bacillus: listeriosis, swine erysipelas, actinomycosis, actinomyces-pyogenes infection disease, corynebacterium disease, atypical acid fast bacillus disease, and pyelonephritis.

Fungus: mycosis.

Protozoa: coccidiosis, toxoplasmosis, and balantidium disease.

Eelworm: swine trichuriasis, swine ascariasis, Osophagostomum dentatum disease, and swine metastrongyle nematode disease.

Arthropod: Sarcoptes scabiei parasitism, and haematopinus suis parasitism and the like.

(iii) In case of applying the invention to a domestic animal house (horse house):

RNA virus: hydrophobia, Japanese encephalitis, equine viral arteriitis, and equine swamp fever.

DNA virus: equine rhinopneumonitis.

Gram-negative aerobic bacillus: glanders, contagious equine metritis.

Gram-negative facultative anaerobic bacillus: equine paratyphoid, and pasteurellosis.

Gram-positive coccus: streptococcus disease, strangles.

Gram-positive spore-forming bacillus: anthrax, and tetanus.

Fungus: epizootic lymphangitis.

Protozoa: trypanosome disease, piroplasmosis, and the like.

(iv) In case of applying the invention to a domestic animal house (cowhouse):

RNA virus: Ibaraki disease, Japanese encephalitis, reovirus disease, bovine rotavirus disease, Chuzan disease, bovine viral diarrhea/mucosal diseases, bovine coronavirus disease, parainfluenza, Metapneumovirus disease, bovine epizootic fever, Akabane disease, ainovirus (phonetic) disease, bovine leukemia, and bovine rhinovirus disease.

DNA virus: bovine papular stomatitis, pseudocowpox, malignant catarrh disease, infectious bovine rhinotracheitis, bovine adenovirus disease.

Rickettsia: anaplasmosis, and epirithlozone (phonetic) disease.

Mycoplasma: mycoplasmosis.

Spirochaeta: leptospirosis.

Spirillum: campylobacter disease,

Gram-negative aerobic bacillus: infectious bovine keratoconjunctivitis, brucellosis, and pseudomonas infectious disease.

Gram-negative facultative anaerobic bacillus: colibacillosis, salmonellosis, pasteurellosis, klebsiella disease, actinobacillus disease, hemophilus/somunus infection disease

Gram-negative anaerobic asporogenic bacillus: fusobacterium necrophorum disease.

Gram-positive coccus: staphylococcus disease, and streptococcus disease.

Gram-positive spore-forming bacillus: anthrax, necrotic enteritis, blackleg, malignant edema, clostridial infection disease, tetanus, and bacillus cereus infectious disease.

Gram-positive asporogenic bacillus: listeriosis, bovine pyelitis, actinomycosis, dermatophilosis, actinomyces-pyogenes infection disease, tuberculosis, paratuberculosis, and corynebacterium disease.

Fungus: mycosis, aspergillus infectious disease, cryptococcosis, and dermatophytosis.

Protozoa: coccidiosis, cryptosporidium disease, neosporosis, piroplasmosis, theileria srgeni infectious disease, and trypanosomiasis.

Eelworm: strongyloides papillosus infection, bovine ascariasis, bovine lungworms infection, and bovine trichuriasis infection.

Cestoid: moniezia benedeni infection disease.

Trematode: paragonimiasis, and amphistome infectious disease.

Arthropod: bovine mange infectious disease,

bovine biting lice, warble infestation infectious disease, demodectic mange and the like.

(v) In case of applying the invention to buildings for human being such as accommodation units:

[First Class Infectious Diseases]

Ebola virus: Ebola hemorrhagic fever.

Crimean-Congo virus: Crimean-Congo hemorrhagic fever.

Plague bacillus: black plague.

Marburg virus: Marburg hemorrhagic fever.

Lassa virus: Lassa fever

[Second Class Infectious Diseases]

Cholera toxin (CT) elaborated by cholera bacilli: cholera. Dicentili (phonetic)/S.flexneri/S.boydii/S.sonnei: shigellosis.

Salmonella typhoid bacillus/Bacterium paratyphosum: abdominalis/paratyphoid

Poliovirus: acute anterior poliomyelitis (polio).

Corynebacterium diphtheriae: diphtheria.

[Third Class Infectious Diseases]

Enterohemorrhagic Escherichia coli (O 157 and the like): enterohemorrhagic Escherichia coli infectious disease.

[Forth Class Infectious Diseases]

Cyst of Entamoeba histolytica: ameba bloody flux.

Echinococcus (alveolar echinococcus, Echinococcus granulosus): echinococcosis.

Yellow fever virus: yellow fever.

Chlamydia psittaci: psittacosis.

Spirochaeta: febris recurrens.

Hepatitis virus: acute viral hepatitis.

Coxiella burnetii (rickettsia): Q-fever.

Rabies virus: hydrophobia.

Cryptosporidium protozoa: cryptosporidiosis.

Pestiferousness prion protein: Creutzfeldt-Jakob disease.

Group A streptococci: fulminant form severe invasive streptococcal infection.

HIV virus: acquired immune deficiency syndrome.

Coccidioides immitis: coccidioidomycosis.

Giardia lamblia: giardiasis.

Hantaan virus: hemorrhagic fever with renal syndrome.

Meningococcus: epidemic cerebrospinal meningitis.

Rubella virus: congenital rubella syndrome.

Anthrax bacillus: anthrax.

Tsutsugamushi disease rickettsia: Tsutsugamushi disease.

Dengue virus: dandy fever.

Rickettsia japonica: Japanese spotted fever.

Japanese encephalitis virus: Japanese encephalitis.

Botulinum: infant botulism.

Treponema pallidum: syphilis.

Tetanus bacillus: tetanus.

Vancomycin-resistant enterococcal: vancomycin-resistant enterococcal infection.

Hantaviruspulmona.rysyndromevirus: Hantavirus pulmonary syndrome.

Ape-derived B-virus: Cercopithecine herpesvirus (CHV-1).

Genus brucella: brucellosis.

Rickettsia prowazekii: camp fever.

Malaria parasite: malaria.

Lyme fever Borrelia: lyme fever.

Legionella bacteria: legionaires' disease.

West Nile virus: West Nile fever.

Adenovirus type 3.7 and the like: pharyngoconjunctival fever.

Group A hemolytic streptococcus: group A hemolytic streptococcus adenoiditis.

Virus (rotavirus, small round structured virus and the like)/Bacteria (salmonella, campylobacter and the like): pestiferousness gastroenteritis.

Varicella-zoster virus: chicken pox. Coxsackievirus and the like: hand-foot-and-mouth disease.

Human parvovirus: erythema infectiosum.

Human herpesvirus: sixth disease.

Bordetella pertussis: pertussis.

Rubella virus: epidemic roseola (three-day measles).

Group A coxsackievirus: herpangina.

Rubeola virus; Measles mumpus virus: epidemic parotiditis.

Enterovirus type 70 and the like: acute hemorrhagic conjunctivitis.

Adenovirus 8 and the like: contagious conjunctivitis (pink-eye).

Protopathic (herpes simplex virus and the like)/Secondary (rubeola virus and the like): acute encephalitis.

Haemophilus influenzae/pneumococcus/meningococcus: bacterial meningitis.

Coxsackievirus and the like: abacterial meningitis.

Mycoplasma pneumoniae: mycoplasma pneumonia.

Chlamydia: chlamydia Pneumoniae.

Acute rubeola virus: adult linen canvas.

Chlamydia trachomatis: genital chlamydia.

Herpes simplex virus: genital herpesvirus infection.

Human papillomavirus: pointed condyloma.

Neisseria gonorrhoeae: gonococcal infection.

MRSA: methicillin-resistant staphylococcus aureus infection.

Penicillin-resistant Streptococcus pneumoniae: penicillin-resistant Streptococcus pneumoniae infection.

Multiple drug resistant pseudomonas aeruginosa: drug-resistant pseudomonas aeruginosa infection.

Tubercle bacillus: tuberculosis.

In addition to the above, new type infection diseases such as severe acute respiratory syndrome (SARS) with SARS corona virus. Furthermore, it is considered that the present invention is also effective for prevention of secondary infections to human being from that derived from animals.

According to the present invention, when cypress oil is allowed to diffuse into the atmosphere inside a domestic livestock house, it becomes possible to effectively prevent infections in living organisms (domestic fowls, domestic animals, and human beings) due to antipathogenic activity derived from extracts of vegetable organisms of cupressaceae as represented by cypress oil and the like. Particularly, it is observed that cypress oil exhibits anti-virus activity, especially high anti-influenza virus activity, so that a variety of virus infections including avian influenza, animal (swine and the like) influenza, and human influenza infections can be very effectively prevented. Accordingly, when the antipathogenic domestic livestock house of the invention is applied to domestic fowl houses, domestic animal houses, and accommodation units and the like, it becomes possible to effectively avoid tremendous damages as resulted from recent troubles of highly pathogenic avian influenza.

### BRIEF DESCRIPTION OF THE DRAWINGS

FIG. 1 is a schematic view illustrating a chicken house being a barn-door fowl house according to a first embodiment of the present invention;

FIG. 2 is a schematic view illustrating a pig house being a livestock barn according to a second embodiment of the present invention;

FIG. 3 is a schematic view illustrating a house according to a third embodiment. of the present invention;

FIG. 4 is a schematic view illustrating the chicken house being a barn-door fowl house according to the first embodiment of the present invention;

FIG. 5 is a schematic view illustrating an essential part of the chicken house being a barn-door fowl house according to the first embodiment of the present invention;

FIG. 6 is a schematic view showing a structure of a disinfectant for chicken according to the first embodiment of the present invention;

FIG. 7 is a schematic view illustrating the pig house being a livestock barn according to the first embodiment of the present invention;

FIG. 8 is a schematic view illustrating a part of the pig house being a livestock barn according to the first embodiment of the present invention.

### DESCRIPTION OF THE PREFERRED EMBODIMENTS

Preferred embodiments of the present invention will be described hereinafter by referring to the accompanying drawings.

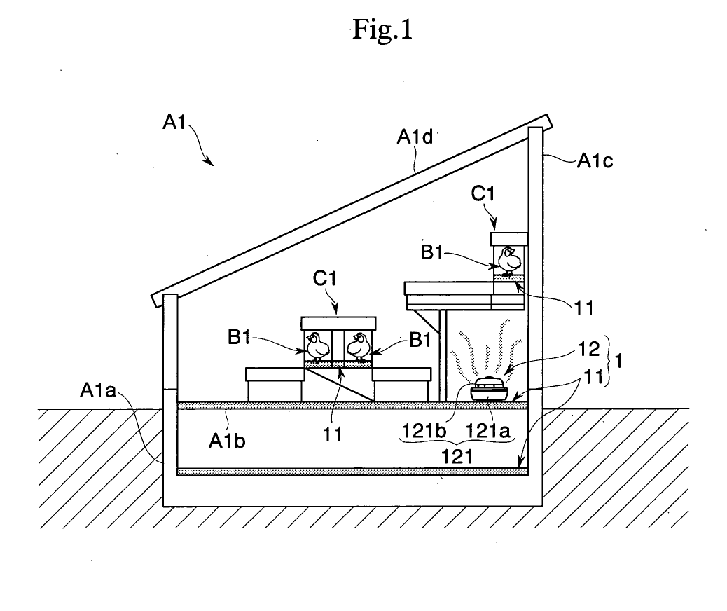

FIG. 1 shows schematically a structure of a barn-door fowl house (hereinafter referred to as “chicken house” for indicating the barn-door fowl house for breeding chickens in the present example) A1 being an example of an antipathogenic domestic livestock house according to the first embodiment of the invention. The chicken house A1 is a building of a usual chicken house which is built on a ground of a chicken yard as a thing firmly affixed and composed by a makeup of a basement A1a, a foundation, a floor A1b, columnae, walls A1c, a roof (ceiling) A1d and the like. Inside the chicken house A1, breeding shelves C1 for breeding chickens B1 in multiply layered shelves are provided. It is, however, noted that the invention is not limited to such construction as mentioned above, but a single-layered shelf may be used, or no such shelf may be provided, in other words, chickens may be subjected to flat rearing. Furthermore, the breeding shelf C1 may be provided with a means for collecting eggs (not shown). On one hand, the chicken house A1 may be provided with a variety of devices such as air-conditioning equipment, feeding and water supply equipment and the like.

The chicken house A1 may be provided with a spreading means 1 for spreading an extracted oil of vegetable organisms of cupressaceae into the internal air, i.e., the atmosphere in the chicken house B1. In the present embodiment, Aomori cypress is applied as a vegetable organism of cupressaceae wherein cypress oil is used as its extracted oil. Such cypress oil is obtained by applying, for example, a usual steam distillation technique wherein a sawdust produced at the time of lumber sawing of Aomori cypress is collected, a distillation still is charged with the sawdust, and the sawdust is made tobe in contact with a steam heated by a boiler, whereby a liquid is extracted and from which an oil component is taken out. In this case, about 1 Kg of cypress oil is obtained from about 100 Kg of Aomori cypress material. The liquid obtained by a steam distillation technique contains cypress extraction water containing water-soluble components dissolved therein other than the cypress oil wherein a weight ratio of the cypress oil and the cypress extraction water is about 1:100. The resulting cypress oil contains about 3 to 4% of acidic oil and about 96 to 97% of neutral oil in weight ratio. The acidic oil contains compounds such as carbachol, 1-rosin acid, hinokitiol, and β-dolabrin, while the neutral oil contains compounds such as thujopsene, p-cymene, dihydrocymene, cedrol, and widdrol.

In the present embodiment, the spreading means 1 uses the following two modes wherein a first mode is a spreading means 11 for using cypress oil adsorbed by a porous material, while a second mode is a spreading means 12 for atomizing misty cypress oil from an atomizer. In the chicken house A1, although only either of the first spreading means 11 and the second spreading means 12 may be used as the spreading means 1, both the first and second spreading means 11 and 12 may also be used as in the present embodiment. Firstly, the first spreading means 11 will be described. In the first spreading means 11, woody pellets being chips of a wood are used as a porous material (examples are illustrated by half-tone dot-meshed regions in the accompanying drawings, and so forth). Such woody pellets are made from, for example, woods such as Japan cedar, and Japanese cypress. However, the invention is not limited to these woods. The porous material is not limited to woody pellets, but it is also possible to properly use a porous material such as activated carbon, charcoals of wood charcoal and bamboo charcoal and the like, powder or milled powder (fine particles) of zeolite ores, ceramic particles, milled powder or powder of diatomaceous earth, porous natural or artificial products, porous organic or inorganic substances, or the mixtures thereof.

A number of opened fine pores exist on the surface of a wood pellet, and cypress oil is adsorbed in the opened fine pores. The first spreading means 11 is constituted by sparging or disposing such cypress oil adsorbed with wood pellets under the floor A1 (on the basement A1a), on the floor A1b, the surface of a breeding shelf C1 and the like of the chicken house A1. It is not necessarily required to sparge or dispose the cypress oil adsorbed with wood pellets on all the places as mentioned above, but it is sufficient to sparge or dispose the cypress oil adsorbed with wood pellets on any of them. In this respect, such cypress oil adsorbed with wood pellets may also be sparged or disposed optionally any places other than that as mentioned above dependent upon a size or its internal atmosphere of the chicken house A1. Cypress oil to be adsorbed with wood pellets may be an extracted oil itself (namely, 100% cypress oil) obtained by steam distillation of Aomori cypress, or a mixed liquor (diluted suspension) of the cypress oil and water or cypress oil extraction water may also be used, and further a diluted aqueous solution obtained by adding an emulsifier to the mixed liquor may be also used. Other diluted liquors prepared by using a suitable solvent (an alcohol and the like having lipophilic nature) may be used also. In this case, a dilution rate may be properly determined. In the present embodiment, a total volume fraction about 60% of an aqueous cypress oil solution (a suitable amount of an emulsifier: a mixture of a cationic material and an anionic material is added) is used. The most simple manner for preparing cypress oil adsorbed with wood pellets is desirably in such that a sieve or the like containing a number of wood pellets is immersed in a vessel in which an aqueous cypress oil solution is accumulated for a predetermined period of time, thereafter, the sieve or the like is taken out from the vessel, and dried up to a certain degree by means of sun drying or artificial drying.

On the other hand, a suitable atomizer 121 composed of a container section 121a for accumulating liquid cypress oil and an atomizing section 121b for atomizing the cypress oil into the air from the container section 121a is used as the second spreading means 12. The atomizer 121 may be provided with a means for adjusting an atomization amount and a timer means (both of them are not shown). A plurality of such atomizers 121 may be disposed in the chicken house A1 in response to atomization ability of the-atomizer. Any form of cypress oil such as a stock solution of extracted oil, a diluted suspension of the extracted oil with water or cypress oil extraction water, a diluted aqueous solution prepared by adding further an emulsifier thereto, a diluted liquor of the extracted oil with a suitable solvent and the like is applicable to the cypress oil to be accumulated in the container section 121a as in the case of the cypress oil adsorbed with wood pellets. In the present embodiment, however, a total volume fraction about 60% of an aqueous cypress oil solution (a suitable amount of an emulsifier: a mixture of a cationic material and an anionic material is added) is used.

As described above, since the spreading means 1 including the first spreading means 11 and the second spreading means 12 are provided, in the chicken house A1 of the present embodiment, the cypress oil is vaporized gradually from the wood pellets of the first spreading means 11 to be discharged into the atmosphere, while the cypress oil is atomized from the atomizer 121 of the second spreading means 12, so that the vaporized cypress oil is spread in the air inside the chicken house A1, whereby the vaporized cypress oil expand around chickens B1. As a result, when cypress oil diffused in the air is taken breath by a chicken B1 or adheres on a surface of the chicken body B1, antipathogenic effects due to cypress oil appear. Thus, infectious disease in a chicken B1, infectious disease in human being who works in the chicken house A1, secondary infection of a pathogenic organism in human being from a chicken B1, infection in a chicken B1 due to a pathogenic organism having a possibility carried by human being into the chicken house A1 and the like infection can effectively be prevented. Since wood pellets used as the first spreading means 11 are made from wood, even if a chicken B1 picks the wood pellets, there is no problem in health. Moreover, it may be considered that even if such wood pellets to which poultry manure adheres that involves a possibility of introduction of pathogenic organisms are picked by a chicken B1, the wood pellets exhibit sufficient effects upon infection control of pathogenic organism, because the wood pellets contain cypress oil. Particularly, it is recognized that cypress oil exhibits remarkable anti-influenza virus activity as in the example which will be mentioned later. Accordingly, it is possible to prevent infection with influenza viruses including highly pathogenic avian influenza and salmonella in a chicken B1 or human being at high percentage in the chicken house A1 of the present embodiment.

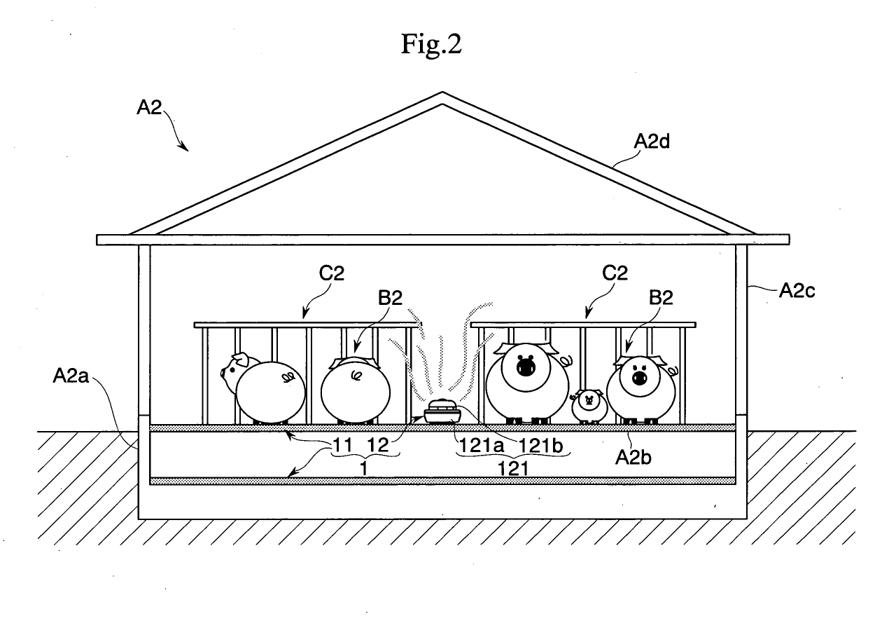

FIG. 2 shows schematically a structure of a livestock barn (hereinafter referred to as “pig house” for indicating the livestock barn for breeding pigs in the present embodiment) A2 being an example of an antipathogenic domestic livestock house according to the second embodiment of the invention. The pig house A2 is a building of a usual pig house which is built on a ground of a hog yard as a thing firmly affixed and composed by a makeup of a basement A2a, a floor A2b, columnae, walls A2c, a roof (ceiling) A2d and the like. Inside the pig house A2, breeding fences C2 for breeding pigs B2 are provided. It is, however, noted that the invention is not limited to such construction as mentioned above, but no such fence may be provided, in other words, pigs may leave loose. Furthermore, the pig house A2 may be provided with a variety of devices such as air-conditioning equipment, feeding and water supply equipment and the like.

In also the present embodiment, a first spreading means 11 and a second spreading means 12 are applied as a spreading means 1 as in the case of the chicken house A1 according to the first embodiment. More specifically, cypress oil adsorbed with wood pellets is disposed or sparged under the floor (on the basement A2a) or on the floor A2b as the first spreading means 11. In addition, liquid cypress oil is atomized into the air inside the pig house A2 from an atomizer 121 provided on the floor A2b as the second spreading means 12. It is to be noted that either of the first spreading means 11 and the second spreading means 12 may be applied as the spreading means 1 in also the present embodiment, and it is the same as in the case of the first embodiment that positions of respective spreading means to be disposed are not limited.

As described above, since the spreading means 1 provided in the pig house A2 of the present embodiment, the cypress oil is vaporized gradually from the wood pellets of the first spreading means 11 to be discharged into the atmosphere, while the cypress oil is atomized from the atomizer 121 of the second spreading means 12, so that the vaporized cypress oil is spread in the air inside the pig house A2, whereby the vaporized cypress oil expand around pigs B2. As a result, when cypress oil diffused in the air is taken breath by a pig B2 or adheres on a surface of the pig body B2, antipathogenic effects due to cypress oil appear. Thus, infectious disease in a pig B2, infectious disease in human being who works in the pig house A2, secondary infection of a pathogenic organism in human being from a pig B2, infection in a pig B2 due to a pathogenic organism having a possibility carried by human being into the pig house A2 and the like infection can effectively be prevented. Since wood pellets used as the first spreading means 11 are made from wood, even if a pig B2 puts into the mouth the wood pellets together with a feeding stuff, there is no problem in health. Moreover, it may be considered that even if such wood pellets to which feces that involves a possibility of introduction of pathogenic organisms adheres, the wood pellets exhibit sufficient effects upon infection control of such pathogenic organisms, because the wood pellets contain cypress oil. Particularly, it is recognized that cypress oil exhibits remarkable anti-influenza virus activity as in the examples which will be mentioned later. Accordingly, it is possible to prevent infection with swine influenza virus in a pig B2 or human being at high percentage in the pig house A2 of the present embodiment.

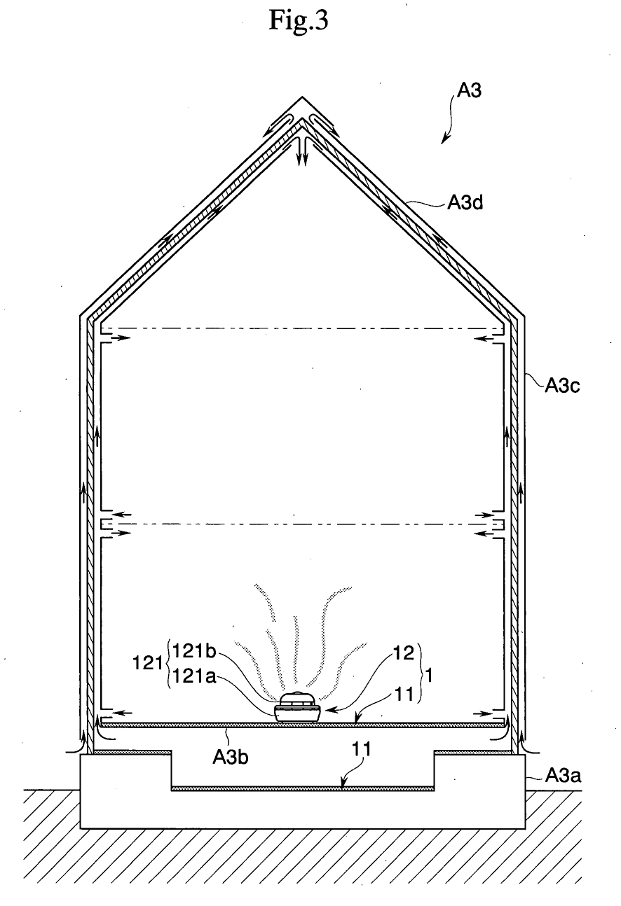

FIG. 3 shows schematically an example of an antipathogenic structure of an accommodation unit (the most usual house for abode in which human being lives is shown in the present embodiment) A3 according to a third embodiment of the invention. The house A3 is a usual building which is built on residential grounds as a thing firmly affixed and composed by a makeup of a basement A3a, a floor A3b, columnae, walls A3c, a roof (ceiling) A3d and the like (the illustrated example is a two-story wooden home) . Although the house A3 is provided with a wall structure for ventilation for circulating air in doors to the walls A3c and the roof A3d, a ventilating structure of a different type may be applied, or a house provided with no such ventilating structure has no problem in the present invention. Furthermore, the invention is not limited to such wooden structure, but it may be a concrete building, and the number of stories is not particularly limited. Accordingly, the invention may be applied to large-scale buildings such as buildings for business uses or communal facilities.

In also the present embodiment, a first spreading means 11 and a second spreading means 12 are applied as a spreading means 1 as in the case of the above-described embodiments. More specifically, cypress oil adsorbed with wood pellets is disposed or sparged under the floor (on the basement A3a) or on the floor A3b as the first spreading means 11. In addition, liquid cypress oil is atomized into the air inside the house A3 from an atomizer 121 provided on the indoor as the second spreading means 12. Particularly, since the wall structure for circulating air of the indoor is applied as in the case of the present invention, vaporized or atomized cypress oil can be widespread all over the interior of the house. It is to be noted that either of the first spreading means 11 and the second spreading means 12 may be applied as the spreading means 1 in also the present embodiment, and it is the same as in the case of the above-described embodiments that positions of respective spreading means to be disposed are not limited.

As described above, since the spreading means 1 provided in the house A3 of the present embodiment, the cypress oil is vaporized gradually from the wood pellets of the first spreading means 11 to be discharged into the atmosphere, while the cypress oil is atomized from the atomizer 121 of the second spreading means 12, so that the vaporized cypress oil is spread in the air inside the house A3, whereby the vaporized cypress oil expand around human being B3. As a result, when cypress oil diffusedintheairis takenbreathbya humanbeingB3oradheres on a surface of the human body B3, antipathogenic effects due to cypress oil appear. Thus, infectious disease in a human being B3, infectious disease in a pet animal bred in the house A3, secondary infection of a pathogenic organism in human being from the pet animal, and the like infection can effectively be prevented. Particularly, it is recognized that cypress oil exhibits remarkable anti-influenza virus activity as in the examples which will be mentioned later. Accordingly, it is possible to prevent infection with human influenza virus in a human being B3 at high percentage in the house A3 of the present embodiment.

It is to be noted that the present invention is not limited to the above-described embodiments, a variety of modifications may be made with respect to types or structures of an antipathogenic domestic livestock house, and specific constructions of a spreading means within a scope of the subject matter of the invention.

Moreover, the invention is not restricted to the antipathogenic domestic livestock house to which only the spreading means as mentioned above is applied.

Next, an antipathogenic integratedly preventing and eliminating system according to a fourth embodiment of the invention will be described in detail. The antipathogenic integratedly preventing and eliminating system is the one for preventing and eliminating complexedly infections in living organisms due to pathogenic organisms by the combination of a sparger 2 being a sparging means which will be described in detail, a disinfectant 3 for a domestic livestock house, a disinfectant 4 for a living organism, a feedstuff 5, and a drinking water 6 in addition to an antipathogenic domestic livestock house A4 according to the present invention.

In the following, the present embodiment will be described in detail by referring to the accompanying drawings wherein the same components as that of the first embodiment are designated by the same reference characters as that of the first embodiment and a detailed explanation therefor will not be repeated here.

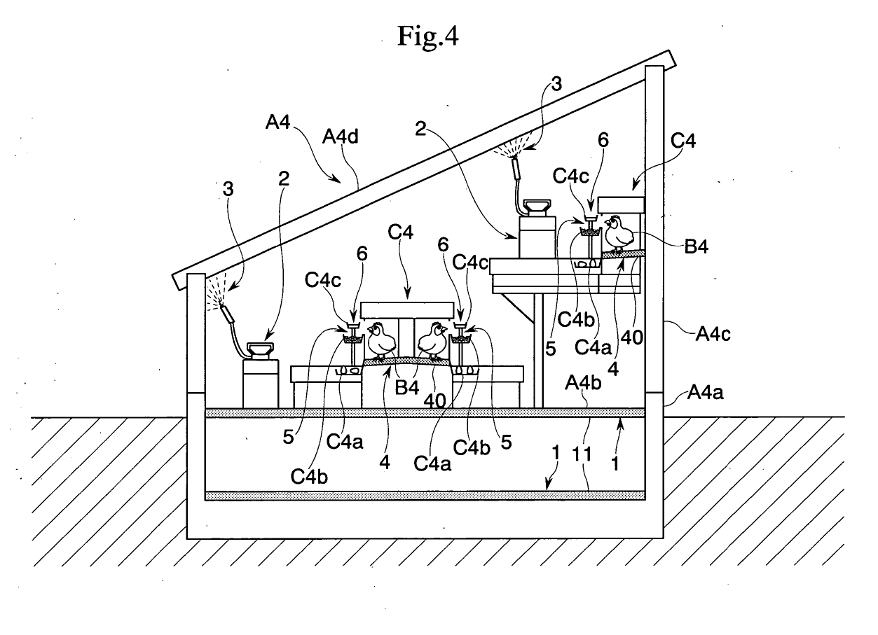

FIG. 4 shows schematically a structure of a barn-door fowl house (hereinafter referred to as “chicken house” for indicating the barn-door fowl house for breeding chickens in the present example) A4 being an example of an antipathogenic domestic livestock house and the internal structure thereof according to a fourth embodiment of the invention. The chicken house A4 is a building of a usual chicken house composed by a makeup of a basement A4a, a foundation, a floor A4b, columnae, walls A4c, a roof (ceiling) A4d and the like. Inside the chicken house A4, breeding shelves C4 for breeding chickens B4 in multiply layered shelves are provided. The disinfectant 4 being a disinfectant for living organisms according to the invention is disposed under a breeding shelf C4. The breeding shelf C4 is provided with an egg collection shelf C4a for collecting eggs, and a feed chute C4b and a water chute C4c constituting feeding and water supply equipment. Accordingly, the present embodiment is practiced in such a mode that since the feeding and water supply equipment. (not shown) is provided in the chicken house A4, the feedstuff 5 and the drinking water 6 for animals according to the present embodiment are supplied in a predetermined amount at a predetermined time which have been previously set up to implement feeding and water supply. It is, however, noted that the chicken house A4 of the invention is not limited to such construction as mentioned above, but a single-layered breeding shelf may be used, or no such shelf may be provided, in other words, chickens may. be subjected to flat rearing.

The chicken house A4 may be provided with the sparger 2 for sparging the disinfectant 3 for a domestic livestock house containing an extracted oil of vegetable organisms of cupressaceae into the internal air, i.e., the atmosphere in the chicken house A4 of the roof (ceiling) A4d, the walls A4c, and the floor A4b. Furthermore, the disinfectant 4 being the one for living organisms is disposed on a breeding shelf C4, and in addition, the feedstuff 5 is disposed in the feed chute C4b and the drinking water 6 being the one for drinking water of animals is disposed in the water chute C4b.

In the present embodiment, Aomori cypress is applied as a vegetable organism of cupressaceae wherein cypress oil is used as its extracted oil to the disinfectant 3 for domestic livestock house, the disinfectant 4, the feedstuff 5, and the drinking water 6 as in the case of the above-described embodiments. Such cypress oil is obtained by applying, for example, a usual steam distillation technique wherein a sawdust produced at the time of lumber sawing of Aomori cypress is collected, a distillation still is charged with the sawdust, and the sawdust is made to be in contact with a steam heated by a boiler, whereby a liquid is extracted and from which an oil component is taken out.

In the following, the sparger 2, the disinfectant 3 for domestic livestock house, the disinfectant 4, the feedstuff 5, and the drinking water 6 according to the present embodiment, respectively, will be described in detail.

A sparger 2 is disposed for sparging the disinfectant 3 for domestic livestock house inside the chicken house A4, i.e. the floor A4b, the walls A4c, and the roof (ceiling) A4d in the present embodiment as illustrated schematically in FIG. 4. The sparger 2 is composed of a tank being charged with the disinfectant 3 for domestic livestock house, a jet head for jetting the disinfectant 3 for domestic livestock house, and a hose for linking the jet head to the tank. It is, however, to be noted that the sparging means according to the invention is not limited to the mode illustrated in the accompanying drawing, but, for example, such a mode wherein a sprinkler is disposed in the chicken house, and the disinfectant 3 for domestic livestock house is sparged, for example, periodically by means of the sprinkler is also applicable.

The disinfectant 3 for domestic livestock house is sparged to the floor A4b, the walls A4c, and the roof (ceiling) A4d from the above-mentioned sparger 2. Moreover, a mode for applying the disinfectant for domestic livestock house is not restricted to the one for sparging the disinfectant inside the chicken house A4, but it is also possible to use the disinfectant 3 for domestic livestock house, for example, in order to disinfect the bottoms of worker's shoes with the use of the disinfectant 3 for domestic livestock house contained in a vat which has been previously prepared at an entrance or the like of the chicken house A4.

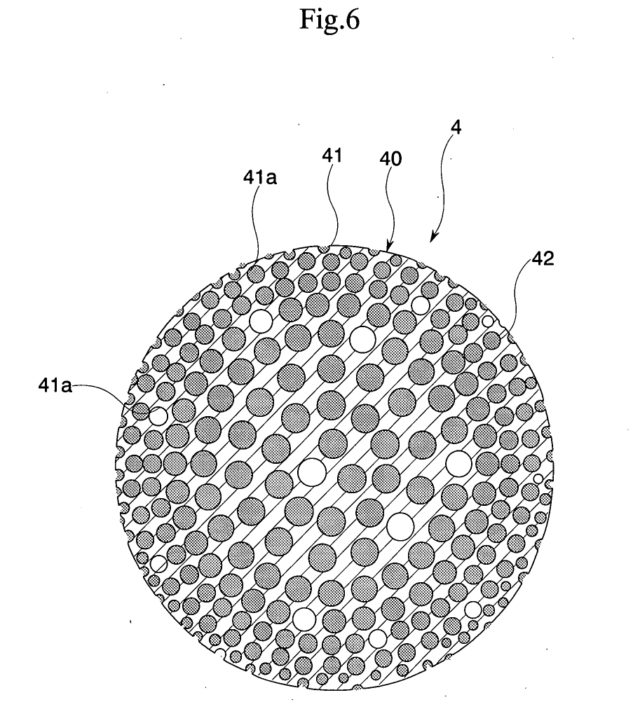

The disinfectant 4 being the one for living organisms is used in the form of a granular disinfectant (hereinafter referred to as simply “pellet”) 40. In more detail, the pellet 40 is composed of diatomaceous earth 41, as its major component, and cypress oil 42 which is allowed to contain into fine pores 41a of the diatomaceous earth 41 as shown in FIG. 6. A single grain of the diatomaceous earth 41 is an ultraporous fine particle having a size of about 50 micrometer diameter and including infinitely many fine pores each having about 0.1 to 0.2 micrometer diameter, for example, as shown in FIG. 6. One of methods for manufacturing the granular disinfectant 40 will be simply described herein. First, a container containing cypress oil (not shown) is charged with a predetermined amount of the diatomaceous earth 41. The cypress oil is, of course, an oil soluble component containing a large amount of hinokitiol in a liquid prepared by applying a high-temperature and a high-pressure steam to wood chips, barks of a tree, foliage and the like which are cut into pieces of the above-mentioned Aomori cypress or Taiwan cypress. Although the diatomaceous earth 41 floats on the liquid surface of the cypress oil at the time of initial charge of the diatomaceous earth 41, it settles down gradually on the bottom of the container after the fine pores 41a of the diatomaceous earth 41 are adsorbed with the cypress oil. When the material so settled down is taken out to obtain the pellets 40 of the present embodiment. The disinfectant for living organisms according to the present invention includes, for example, a liquid formulation containing cypress oil for washing the body of a chicken B4 which is introduced newly into the chicken house A4 from the outside thereof other than the pellet 40 as mentioned above.

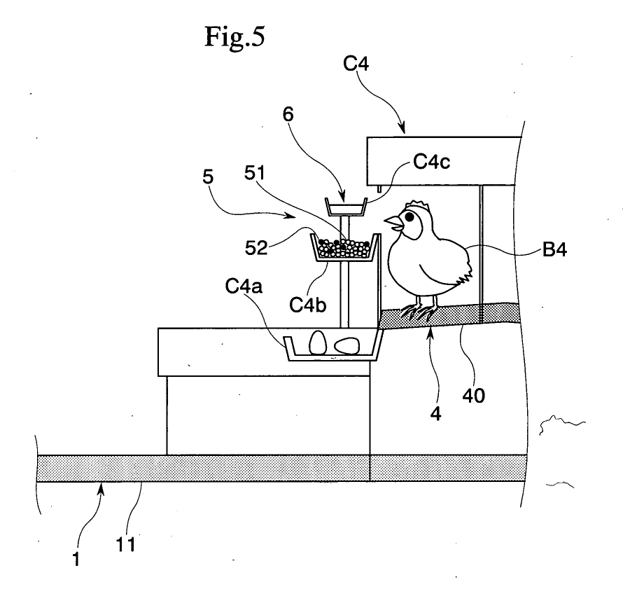

As shown in FIG. 5, the feedstuff 5 of the present embodiment is a mixture of a main feedstuff 51 for chicken usually applied and a suitable amount of a feed additive 52 containing cypress oil. The constitution of the feedstuff according to the invention is not limited to those described above, but a variety of constitutions, for example, a feedstuff prepared by suitably admixing a liquid or powder feed additive with the main feedstuff 51 for containing cypress oil as a whole of the feedstuff may be applied.

The drinking water 6 is the one prepared by adding cypress oil having a predetermined concentration to water which is usually drunk by a chicken B4. In this case, of course, a component other than cypress oil may be added to the water, or either cypress oil may be replaced by cypress oil extraction water or added further such cypress oil extraction water to cypress oil.

As described above, since the antipathogenic integratedly preventing and eliminating system is applied to the present embodiment, the disinfectant 3 for domestic livestock house is sparged to the walls A4c, the floors A4b, and the roof (ceiling) A4d of the chicken house A4 by means of the sparging means 2. Thus, a propagation of pathogenic organisms is prevented in the chicken house A4 being the antipathogenic domestic livestock house, and further the cypress oil is vaporized gradually fromthe woodpellets of the first spreading means 11 to be discharged into the atmosphere, so that the vaporized cypress oil is spread in the air inside the chicken house A4, whereby the vaporized cypress oil expand around chickens B4. Besides, the chickens B4 eat the feedstuff 5 containing cypress oil, and drink the drinking water 6 containing also cypress oil. As a result, when cypress oil diffused in the air is taken breath by the chickens B4 or adheres on a surface of the chicken bodies B4, antipathogenic effects due to cypress oil appear complexedly. Thus, infectious disease in the chickens B4, infectious disease in human being who works in the chicken house A4, secondary infection of a pathogenic organism in human being from the chickens B4, infection in the chickens B4 due to a pathogenic organism having a possibility carried by human being into the chicken house A4 and the like infection can effectively be prevented severalfold. Particularly, it is known that when an influenza virus invades in the body of a chicken B4, the virus proliferates in its enteric canal, even if the virus is hypovirulent. In this respect, when a chicken B4 takes the feedstuff 5 and the drinking water 6 containing cypress oil into its body, it results in a direct prevention of proliferation of a pathogenic organism, particularly influenza virus in the chicken's (B4) enteric canal. Moreover, even when a chicken B4 puts wood pellets used as the first spreading means 11 and pellets 40 used as a disinfectant for living organisms into its mouth, it may be considered that there is a sufficient infection control effect of pathogenic organisms, since the wood pellets and the disinfectant pellets 40 contain the cypress oil 42 (FIG. 4). In addition, it is confirmed that cypress oil has antiseptic effects with respect to not only influenza virus, but also salmonella. Accordingly, it may be concluded that when the antipathogenic integratedly preventing and eliminating system of the invention is applied to the chicken house A4 as mentioned above, infection derived from salmonella can be also complexedly prevented.

Furthermore, cypress oil exhibits remarkable resistance to avian influenza virus activity as in the examples which will be mentioned later. Accordingly, when a part or the whole of the pathogenic organism integratedly preventing and eliminating system consisting principally of the antipathogenic domestic livestock house according to the present embodiment is applied, it is possible to prevent infections of influenza viruses including highly pathogenic avian influenza virus or salmonella in the chickens B4 or human being at high percentage.

As described above, when the sparger 2 being a sparging means is provided in the chicken house A4, pathogenic organisms adhere to the inside of the chicken house A4 being a domestic livestock house can be effectively deadened, and in addition, infections in the chickens B4 inside the chicken house A4 can be effectively prevented.

As a result of using the disinfectant 3 for domestic livestock house which contains cypress oil, it is possible to prevent effectively infections of influenza viruses or salmonella in the chickens B4 from the chicken house A4 and it is the same as the above-described embodiments.

Furthermore, since the pellets 40 are used as the disinfectant 4, it is possible to effectively prevent adherence of pathogenic organisms to the body of a chicken B4. Even if a pathogenic organism exists in feces of a chicken B4, infection in others may be effectively prevented, when the feces are in contact with the pellets 40 disposed under the chickens B4.

Besides, since the feedstuff 5 and the drinking water 6 containing cypress oil which is for animal use are used, the cypress oil may be taken in a chicken B4 through its mouth being a principal infection route, i.e. oral infection of influenza viruses, salmonella and the like. Thus, such infections can be effectively prevented and eliminated in accordance with the present embodiment.

The antipathogenic integratedly preventing and eliminating system is not only applied to the above-described chicken house A4.

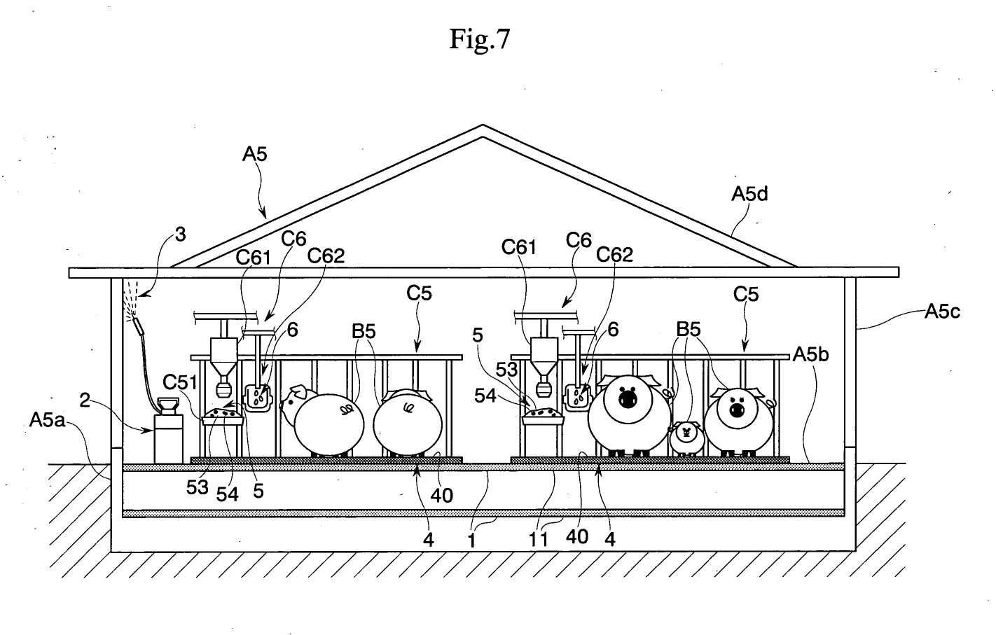

FIG. 7 shows schematically a structure of a livestock barn (hereinafter referred to as “pig house” for indicating the livestock barn for breeding pigs in the present embodiment) A5 being an example of an antipathogenic domestic livestock house, and an example wherein an antipathogenic integratedly preventing and eliminating system is introduced into the pig house according to a fifth embodiment of the invention. The pig house A5 is a building of a usual pig house which is built on a ground of a hog yard as a thing firmly affixed and composed by a makeup of a basement A5a, a floor A5b, columnae, walls A5c, a roof (ceiling) A5d and the like. Inside the pig house A5, breeding fences C5 for breeding pigs are provided. Each of the breeding fences C5 is provided with a feed dish C51 for supplying a feedstuff which will be mentioned later, and under the fence C5, pellets 40 are disposed as a disinfectant 4 as in the case of the fourth embodiment. It is, however, noted that the invention is not limited to such construction as mentioned above, but no such fence may be provided, in other words, pigs may leave loose. Moreover, the pig house A5 is provided with feeding and water supply equipment C6 composed of a feeding device C61 for feeding a feedstuff 5, for example, by a predetermined amount at a predetermined time and a water supplying device C62 for supplying water 6 by a predetermined amount at a predetermined time.

The pig house A5 is provided with the sparger 2 for sparging the disinfectant 3 for a domestic livestock house containing an extracted oil of vegetable organisms of cupressaceae into the internal air, i.e., the atmosphere in the pig house A5 of the roof (ceiling) A5d, the walls A5c, and the floor A5b. Furthermore, pellets 40 are disposed as the disinfectant 4 being the one for living organisms as mentioned above on a breeding shelf C4, and in addition, the feedstuff 5 is disposed in the feeding device C61 and the feed dish C51 and the drinking water 6 being the one for drinking water of animals is disposed in the water supplying device C62.

In the following, the sparger 2, the disinfectant 3 for domestic livestock house, the disinfectant 4, the feedstuff 5, and the drinking water 6 according to the present embodiment will be described. The sparger 2 is disposed for sparging the disinfectant 3 for domestic livestock house inside the pig house A5, i.e. the floor A5b, the walls A5c, and the roof (ceiling) A5d in the present embodiment as illustrated schematically in FIG. 7. It is, however, to be noted that the sparging means according to the invention is not limited to the mode illustrated in the accompanying drawing, but, for example, such a mode wherein a sprinkler is disposed in the pig house, and the disinfectant 3 for domestic livestock house is sparged, for example, periodically by means of the sprinkler is also applicable. The disinfectant 3 for domestic livestock house is spargedto the floor A5b, the walls A5c, and the roof (ceiling) A5d from the above-mentioned sparger 2, to suppress proliferation of pathogenic organisms inside the pig house A5. The disinfectant 4 being the one for living organisms is used as the pellet 40 in the present embodiment. In more detail, the granular disinfectant 40 which is used as the disinfectant 4 for living organisms is composed of diatomaceous earth 41, as its major component, and cypress oil 42 which is allowed to contain into fine pores 41a of the diatomaceous earth 41 as shown in the above FIG. 6. As the disinfectant for living organisms according to the present embodiment, sawdust containing extracts of vegetable organisms of cupressaceae such as cypress oil adsorbed with sawdust may be used other than the above-mentioned pellets 40. As shown in FIG. 5, the feedstuff 5 of the present embodiment is a mixture of a main feedstuff 53 for pig usually applied and a suitable amount of a feed additive 54 containing cypress oil. The drinking water 6 is the same as the one prepared in the case of the fourth embodiment by adding cypress oil having a predetermined concentration to water which is usually drunk by a pig B5.

As described above, since the antipathogenic integratedly preventing and eliminating system is applied to the present embodiment, the disinfectant 3 for domestic livestock house is sparged to the walls A5c, the floors A5b, and the roof (ceiling) A5d of the pig house A5 by means of the sparging means 2. Thus, a propagation of pathogenic organisms is prevented in the pig house A5 of the present embodiment, and further the cypress oil is vaporized gradually from the wood pellets of the first spreading means 11 to be discharged into the atmosphere, so that the vaporized cypress oil is spread in the air inside the pig house A5, whereby the vaporized cypress oil expand around pigs B5. Besides, the pigs B5 eat the feedstuff 5 containing cypress oil, and drink the drinking water 6 containing also cypress oil. As a result, when cypress oil diffused in the air is taken breath by the pigs B5 or adheres on a surface of the pig bodies B5 or is taken into the bodies of the pigs B5 by the feedstuff 5 and the drinking water 6, antipathogenic effects due to cypress oil appear complexedly. Thus, infectious disease in the pigs B5, infectious disease in human being who works in the pig house A5, secondary infection of a pathogenic organism in human being from the pigs B5, infection in the pigs B5 due to a pathogenic organism having a possibility carried by human being into the pig house AS and the like infection can effectively be prevented severalfold. Particularly, it is known that pigs are also infected with influenza viruses. In this respect, when a pig B5 takes the feedstuff 5 and the drinking water 6 containing cypress oil into its body, it results in a direct prevention of proliferation of a pathogenic organism, particularly influenza viruses in the pig's (B5) body. Moreover, even when a pig B5 puts wood pellets used as the first spreading means 11 and pellets 40 into its mouth, it may be considered that there is a sufficient infection control effect of pathogenic organisms, since the wood pellets and the disinfectant pellets 40 contain the cypress oil. In addition, it is considered that cypress oil has antiseptic effects with respect to a variety of types of influenza viruses in view of the fact that the cypress oil exhibits a remarkable resistance to avian influenza virus activity in addition to a resistance to human being influenza virus activity as described in the example which will be described later. As described above, when a part or the whole of the pathogenic organism integratedly preventing and eliminating system consisting principally of the pig house A5 being the antipathogenic domestic livestock house according to the present embodiment is applied, it is possible to prevent infections of influenza viruses including highly pathogenic avian influenza virus, salmonella, and the other pathogenic organisms in a pig B5 or human being can be prevented at a high percentage.

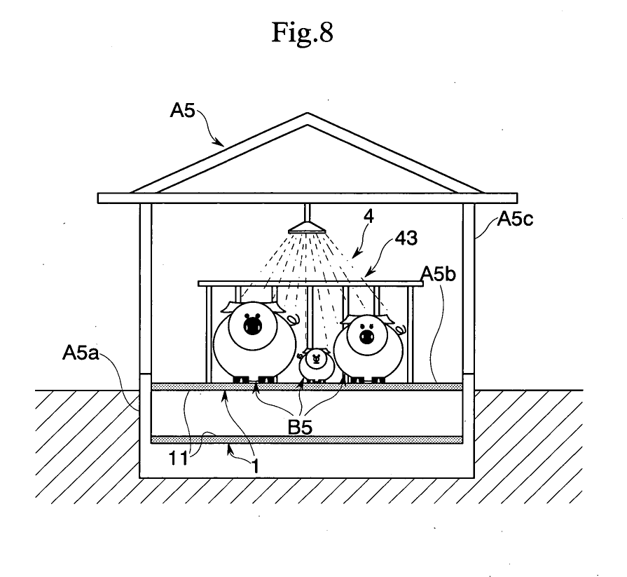

As another mode for applying the disinfectant 4 for a living organism according to the present invention, a liquid formulation 43 being the disinfectant 4 of the invention is disposed at, for example, a front parlor or the like space positioned in the vicinity of an entrance of the pig house A5 as shown in FIG. 8, and the liquid formulation 43 being the one for the disinfectant 4 according to the present invention may be sparged to the pigs B5. When the liquid formulation 43 is applied, it is possible to effectively prevent infections derived from pathogenic organisms which are carried with, for example, pigs' B5 body surfaces from the outside the pig house A5 in case of a new introduction of the pigs B5. It is to be noted that such pathogenic organism integratedly preventing and eliminating systems as mentioned above are also applicable to cow barns and the other domestic livestock houses.

Furthermore, the present invention is not limited to the above-mentioned respective embodiments. More specifically, for example, cypress oil may be replaced by cypress extraction water, or mixtures thereof may be applied as extracts of vegetable organisms of cupressaceae. In addition, one or plural types of compounds containing the above-described components are also applicable. Vegetable organisms other than Aomori cypress may be used so far as they are vegetable organisms of cupressaceae. Irrespective of types of chicken house such as a cage type chicken house, a flat rearing chicken house, and a windowless chicken house, or pig house such as a Danish type pig house, and American type pig house, the invention is applicable. Moreover, it is possible to make a variety of modifications as to specific constitutions of types, structures, and spreading means of the antipathogenic domestic livestock house according to the invention so far as they are not departed from the subject matter of the present invention.

### EXAMPLES

The invention will be described in more detail hereinafter in conjunction with examples. It is, however, to be noted that the present invention is not restricted by the following description. The following respective tests were conducted through a commission to Department of Infectious Diseases of Osaka Prefectural Institute of Public Health.

<Test 1: Comparative Trials of Anti-Influenza Virus Effects of Cypress Derivatives>

(1) Purpose: It is determined which fraction in water-soluble or oil-soluble steam extracts of Aomori cypress exhibits anti-influenza virus activity.

(2) Test specimen: (a) cypress extraction water; a water-soluble component (the lower layer) in extraction liquids obtained by steam distillation of a sawdust powder of Aomori cypress wood material, and specifically, it is 100% cypress extraction water (trade name “Shinrin no Chikara (Force of Forest)” manufactured by Topix Co., Ltd.), (b) cypress oil aqueous solution (60% cypress oil); an oil-soluble component (the upper layer) in the above-described extraction liquids is dissolved in water together with a predetermined amount of an emulsifier. A volume ratio of the cypress oil is 60% of the total amount, and specifically, it is a natural termite exterminator (trade name “Water-soluble Cypress Oil HB-60” manufactured by Topix Co., Ltd.), and (c) hinokitiol copper complex; a copper complex of purified hinokitiol.

The respective test specimens are diluted with a culture fluid at several dilution rates indicated in the following Table 1, and the thus diluted specimens are served for tests.

(3) Test method: Infection tests of influenza viruses according to FFU assay (Focus Forming Unit Assay) are made. Specifically, a predetermined amount of MDCK cells derived from a dog kidney incubated in a microplate with 96 holes (culture fluid: EAGLE MEM) is infected with an influenza virus (H3N2 type human vaccine New Caledonia strain), and after 16 hours, infected cells are stained in accordance with immunostaining technique (1 FFU=1 infectious virus) and measured. A concentration of test specimen wherein the number of infected cells is reduced to 50% is to be 50% Effective Concentration (EC 50). In this case, the influenza virus is kept in a floating state in the culture fluid, and 0.1 ml (the number of survived virus is about 200) thereof is taken in the microplate into which the MDCK cells have been placed.

(4) Test results: Results obtained by determining average values of FFU from three times test upon each test specimen are shown in Table 1.

TABLE 1

| Force of Forest | | Cypress Oil | | Copper Complex | |
|---|---|---|---|---|---|
| Dilution | | Dilution | | Concentration | |
| Rate | FFU | Rate | FFU | (μg/ml) | FFU |
| ×10 | 70 | ×1000 | Exfoliation | 100 | Exfoliation |
| ×50 | 144 | ×5000 | Exfoliation | 20 | Exfoliation |
| ×250 | 171 | ×25000 | Exfoliation | 4 | Exfoliation |
| | | ×125000 | 21 | 0.8 | 280 |
| | | ×625000 | 39 | | |
| Virus | 220 | | | | |
| Only | | | | | |

From the results of the test 1, EC 50 of the cypress oil aqueous solution exhibits a dilution rate of 625,000 times or more (corresponding to a dilution rate of 1,041,667 times or more converted into that of cypress oil formulated concentrate (100% cypress oil)), and accordingly, it is considered that anti-influenza virus activity is significantly high. On the other hand, EC 50 of the cypress extraction water exhibits a dilution rate of about 10 times, and the hinokitiol copper complex is ineffective. The reason of such result that the cypress extraction water exhibits anti-influenza activity is not necessarily clear, but it may be considered that dissolution of acidic oil in the cypress extraction water contributes to the anti-influenza activity though an amount of the acidic oil is an extremely small amount.

<Test 2: Anti-Influenza Virus Effects of Cypress Oil>

(1) Purpose: Since it is found that cypress oil exhibits very high anti-influenza virus activity, differences in infections of an influenza virus with respect to cells dependent upon administration timing of the cypress oil are examined.

(2) Test specimens: Cypress oil (the same cypress oil aqueous solutions as that of the test 1) is used in the respective dilution rates as in the following Table 2.

(3) Test method: Infection tests of an influenza virus in accordance with substantially the same FFU assay as that of the test 1 are conducted with the proviso that (i) a pre-determined amount of incubated MDCK cells (0.1 ml) is added to a microplate to which a culture medium (EAGLE MEM) has been placed, and (ii) to which 0.1 ml of an influenza virus solution (the number of survived viruses is about 200/culture fluid) (the same as that of the test 1) (hereinafter referred to as simply “virus solution”). Then, (iii) the influenza viruses are adsorbed with MDCK cells at 37° C. for 1 hour, and (iv) the virus solution is withdrawn by suction, a culture medium is added thereto and cleaning is carried out by means of suction two or three times, thereafter the MDCK cells are incubated at 37° C. for 16 hours. In this case, four patters of timing as to processing are defined as follows.

“Processing before infection”: A case where cypress oil aqueous solutions in respective dilution rates are added to the microplate in the stage (i) and maintained for a predetermined period of time (10 minutes) until the stage (ii) is started.

“Processing at the time of adsorption”: A case where the cypress oil aqueous solutions in the respective dilution rates are added to the microplate together with the virus solution in the stage (ii).

“Processing after adsorption”: A case where the cypress oil aqueous solutions are added after 16 hours of incubation in the stage (iv).

“Processing at the time of/after adsorption”: A case where the cypress oil aqueous solutions in respective dilution rates are added to the microplate in both the stages (ii) and (iv).

Infected cells are stained in accordance with immunostaining technique (1 FFU=1 infectious virus) and measured in these four patterns of “processing before infection”, “processing at the time of adsorption”, “processing after adsorption”, and “processing at the time of/after adsorption”.

(4) Test results: Results of determined average values of FFU as a result of tests of three times per each test classification are shown in Table 2.

TABLE 2

| Number of | Processing | | | |
|---|---|---|---|---|
| Times in | before | Processing at | Processing | Processing at |
| Dilution | Infection | Time of | after | Time of/after |
| (×10000) | (10 minutes) | Adsorption | Adsorption | Adsorption |
| 10 | 0 | 4 | 183 | 0 |
| 50 | 21 | 8 | 174 | 11 |
| 250 | 54 | 30 | 191 | 34 |
| 1250 | 143 | 95 | 186 | 111 |
| Culture | 187 | | | |
| medium | | | | |

From the results of the test 2, it is found that high anti-influenza virus activity is observed in “processing at the time of absorption” and “processing at the time of/after adsorption”, and EC 50 of them exhibits a dilution rate of about 12,500,000 times (corresponding to a dilution rate of 20,833,333 times converted into that of cypress oil formulated concentrate). On one hand, anti-influenza virus activity is scarcely observed in “processing after adsorption”. Furthermore, substantially equal anti-influenza virus activity in the “processing at the time of/after adsorption” is observed in the “processing before infection”. From the results as described above, it may be recognized that cypress oil exhibits infection inhibitive activity to cells against influenza virus in the case where the influenza virus is adsorbed with cells or before that when the influenza virus is in contact with the cells. Accordingly, it is considered that the cypress oil has an adsorption inhibitive action in cells against influenza virus. Although a functional mechanism therefor is not necessarily clear, there is such a possibility that cypress oil functions upon a side of cells, whereby a step after adsorption of an influenza virus with the cells is inhibited. Accordingly, it is considered that the principal infection inhibitive mechanism relates to adsorption inhibition.

<Test 3: Tests for Studying Processing Time Before Infection>

(1) Purpose: In view of the results in the test 2, a processing time before infection wherein infection inhibitive effects of cells due to cypress oil against influenza viruses are observed is studied.

(2) Test method: Tests are conducted in accordance with the method of the test 2. In the present tests, however, a time when cypress oil is added to a cell cultural fluid is prior to addition of an influenza virus to the cell cultural fluid. In this connection, a plurality of manners is varied as indicated in the following Table 3 within a “processing time before infection” extending a period of time from addition of cypress oil to addition of an influenza virus.

(3) Test results: Results of determined average values of FFU as a result of tests of three times per each test classification are shown in Table 3. For the comparison, FFU values in the “processing at the time of adsorption” (see the test method in the test 2) wherein an influenza virus is added at the same time of cypress oil aqueous solution to a cell cultural fluid are also measured.

TABLE 3

| Number of | | | | | |
|---|---|---|---|---|---|
| Times in | Processing before Infection | | | | |
| Dilution | Two | Five | Fifteen | Thirty | Processing at Time |
| (×10000) | minutes | minutes | minutes | minutes | of Adsorption |
| 10 | 225 | 35 | 16 | 32 | 13 |
| 50 | 227 | 110 | 65 | 60 | 14 |
| 250 | 259 | 178 | 153 | 93 | 24 |
| 1250 | 243 | 264 | 197 | 151 | 132 |
| Culture | 285 | | | | |
| medium | | | | | |

From the results of the test 3, it is found that two minutes are insufficient, while substantially the same effects as that of the “processing at the time of adsorption” are observed in thirty minutes with respect to the processing time before infection wherein adsorption (infection) inhibitive effects of cells due to cypress oil against influenza viruses are observed. When the processing time before infection is five minutes, EC 50 exhibits a dilution rate of about 500,000 times of cypress oil aqueous solution (corresponding to a dilution rate of about 833,333 times converted into that of cypress oil formulated concentrate), and when substantially the same effects as that in the “processing at the time of adsorption” are observed at a dilution rate of 100,000 times of cypress oil aqueous solution (corresponding to a dilution rate of about 166,667 times converted into that of cypress oil formulated concentrate). From the results mentioned above, although an infection inhibitive mechanism in cells against influenza virus due to cypress oil is not necessarily clear, it is considered that there is such a possibility that the cypress oil combines with a cell membrane (possibly, an influenza virus receptor or in the vicinity thereof), whereby adsorption of the virus is inhibited. On one hand, it may be also considered that there is such a possibility that cypress oil infiltrates inside a cell to inhibit a proliferating step of an influenza virus in the cell.

<Test 4: Evaluation Test in Cytotoxicity of Cypress Oil>

(1) Purpose: Evaluation of cytotoxicity in cypress oil is made with taking use of an anti-influenza viral drug derived from cypress oil in animals including human being into consideration.

(2) Test specimen: Cypress oil (The same cypress oil aqueous solutions as those of the tests 1, 2, and 3) is used with proper respective dilution rates.

(3) Test method: Cypress oil is added to a cultural fluid to incubate MDCK cells for 16 hours (wherein the same cultural fluids and cells as that of the tests 1 to 3 are used), and then, an enzyme activity of mitochondria is measured in accordance with MTT method.

(4) Test results: At a dilution rate of 25,000 times of cypress oil aqueous solution (corresponding to a dilution rate of about 41,667 times converted into that of cypress oil formulated concentrate), the enzyme activity decreases to 72%, while cytotoxicity is not observed at a dilution rate of 125,000 times of cypress oil aqueous solution (corresponding to a dilution rate of about 208,333 times converted into that of cypress oil formulated concentrate). Furthermore, when cypress oil aqueous solution is subjected to processing before infection for thirty minutes, cytotoxicity is not observed even at a dilution rate of 1,000 times of cypress oil aqueous solution (corresponding to a dilution rate of about 1,667 times converted into that of cypress oil formulated concentrate).

<Test 5: Comparative Trials In-Effects of Avian Influenza Virus Due to Cypress Derivatives>

(1) Purpose: It is examined whether or not steam extracts of Aomori cypress have resistance to avian influenza activity.

(2) Test specimens: Cypress oil (the same cypress oil aqueous solutions as that of the tests 1, 2, 3, and 4) is used with proper respective dilution rates.

(3) Test method: Infection tests of influenza viruses according to FFU assay (Focus Forming Unit Assay) are made. Specifically, a predetermined amount of MDCK cells derived from a dog kidney incubated in a microplate with 96 holes (culture fluid: EAGLE MEM) is infected with an avian influenza virus (160 FFU, H5N9, type), and after 16 hours, infected cells are stained in accordance with immunostaining technique (1 FFU=1 infectious virus) and measured. An inhibition rate of cypress oil aqueous solution is calculated by the following equation.

$$Inhibition Rate ( \% ) = ( 1 - \frac{\begin{matrix}FFU of a well to which cypress oil having \\ a corresponding concentration is added\end{matrix}}{FFU of a well to which no cypress oil is added} ) ⨯ 100$$

TABLE 4

| Dilution Rate (×10,000) | Inhibition Rate (%) |
|---|---|
| 10 | 100 |
| 50 | 96 |
| 250 | 64 |
| 1250 | 0 |

From the results of the test 5, it is found that a concentration of 90% inhibition effects corresponds to a dilution rate of 500,000 times or more of cypress oil aqueous solution (corresponding to a dilution rate of 833,333 times or more converted into that of cypress oil formulated concentrate (100% cypress oil)) . As described above, it may be considered that cypress oil exhibits significantly high activity with respect to H3 type human influenza virus as well as high resistance to H5 type avian influenza virus activity. Besides, it is considered that there is such a very high possibility that cypress oil exhibits activity against antipathogenic avian influenza virus, since H5 type influenza virus includes antipathogenic influenza viruses.

In accordance with the present invention, when cypress oil is allowed to diffuse into the atmosphere inside a domestic livestock house, it becomes possible to effectively prevent infections in living organisms (domestic fowls, domestic animals, and human beings) due to antipathogenic activity derived from extracts of vegetable organisms of cupressaceae as represented by cypress oil and the like. Particularly, it is observed that cypress oil exhibits anti-virus activity, especially high anti-influenza virus activity, so that a variety of virus infections including avian influenza, animal (swine and the like) influenza, and human influenza infections can be very effectively prevented. Accordingly, when the antipathogenic domestic livestock house of the invention is applied to domestic fowl houses, domestic animal houses, and accommodation units and the like, it becomes possible to effectively avoid tremendous damages as resulted from recent troubles of highly pathogenic avian influenza.

1. An antipathogenic domestic livestock house, comprising:
a building structure for breeding or living a living organism;
extracts of vegetable organisms of cupressaceae for preventing and eliminating pathogenic infections of the living organism; and
a spreading means for spreading the extracts of vegetable organisms of cupressaceae into an atmosphere surrounding the living organism.

2. The antipathogenic domestic livestock house as defined in claim 1, wherein:
the spreading means is a means for sparging or disposing a porous material with which a liquid containing the extracts is adsorbed at least on a floor, under the floor, or a ground of the building structure.

3. The antipathogenic domestic livestock house as defined in claim 1, wherein:
the spreading means is a means for atomizing the liquid containing the extracts inside the building structure by means of a atomizer.

4. An antipathogenic domestic livestock house, comprising:
a building structure for breeding or living a living organism;
a liquid containing extracts of vegetable organisms of cupressaceae for preventing and eliminating pathogenic infections of the living organism; and
a sparging means for sparging the liquid on at least a ceiling, a wall, or a floor in the building structure.

5. The antipathogenic domestic livestock house as defined in claim 1, wherein:
the extracts are extracted oils of vegetable organisms of cupressaceae.

6. The antipathogenic domestic livestock house as defined in claim 5, wherein:
the extracted oils contain lipophilic components in the extracts obtained by steam distillation of a crushed material or powder of vegetable organisms of cupressaceae.

7. The antipathogenic domestic livestock house as defined in claim 1, wherein:
the living organism is a domestic fowl.

8. The antipathogenic domestic livestock house as defined in claim 1, wherein:
the living organism is a domestic animal.

9. The antipathogenic domestic livestock house as defined in claim 1, wherein:
the living organism is human being.

10. A disinfectant for a domestic livestock house, comprising:
extracts of vegetable organisms of cupressaceae served for disinfecting the domestic livestock house for breeding or living a living organism.

11. The disinfectant for a domestic livestock house as defined in claim 10, wherein:
the extracts are extracted oils of vegetable organisms of cupressaceae.

12. The disinfectant for a domestic livestock house as defined in claim 11, wherein:
the extracted oils contain lipophilic components in the extracts obtained by steam distillation of a crushed material or powder of vegetable organisms of cupressaceae.

13. The disinfectant for a domestic livestock house as defined in claim 10, wherein:
the disinfectant is served for suppressing a pathogenic infection in the living organism, and a pathogen of the infection to be suppressed is an influenza virus.

14. The disinfectant for a domestic livestock house as defined in claim 13, wherein:
the pathogen of the infection to be suppressed is an avian influenza virus.

15. The disinfectant for a domestic livestock house as defined in claim 10, wherein:
the disinfectant is served for suppressing a pathogenic infection in the living organism, and a pathogen of the infection to be suppressed is salmonella.

16. A disinfectant for living organisms, comprising:
extracts of vegetable organisms of cupressaceae served for suppressing pathogenic infections of the living organisms.

17. The disinfectant for living organisms as defined in claim 16, wherein:
the disinfectant is composed of, as its principal component, a porous material with which a liquid containing the extracts is adsorbed.

18. The disinfectant for living organisms as defined in claim 17, wherein:
the extracts are extracted oils of vegetable organisms of cupressaceae.

19. The disinfectant for living organisms as defined in claim 18, wherein:
the extracted oils contain lipophilic components in the extracts obtained by steam distillation of a crushed material or powder of vegetable organisms of cupressaceae.

20. The disinfectant for living organisms as defined in claim 16, wherein:
a pathogen of the infection to be suppressed is an influenza virus.

21. The disinfectant for living organisms as defined in claim 20, wherein:
the pathogen of the infection to be suppressed is an avian influenza virus.

22. The disinfectant for living organisms as defined in claim 16, wherein:
the pathogen of the infection to be suppressed is salmonella.

23. The disinfectant for living organisms as defined in claim 16, wherein:
the living organism is a domestic fowl.

24. The disinfectant for living organisms as defined in claim 16, wherein:
the living organism is a domestic animal.

25. A feedstuff, comprising:
extracts of vegetable organisms of cupressaceae served for suppressing pathogenic infections of living organisms.

26. The feedstuff as defined in claim 25, wherein:
the extracts of vegetable organisms of cupressaceae is contained as an additive.

27. The feedstuff as defined in claim 25, wherein:
the extracts are extracted oils of vegetable organisms of cupressaceae.

28. The feedstuff as defined in claim 27, wherein:
the extracted oils contain lipophilic components in the extracts obtained by steam distillation of a crushed material or powder of vegetable organisms of cupressaceae.

29. The feedstuff as defined in claim 25, wherein:
a pathogen of the infection to be suppressed is an influenza virus.

30. The feedstuff as defined in claim 29, wherein:
the pathogen of the infection to be suppressed is an avian influenza virus.

31. The feedstuff as defined in claim 25, wherein:
the pathogen of the infection to be suppressed is salmonella.

32. The feedstuff as defined in claim 25, wherein:
the living organism is a domestic fowl.

33. The feedstuff as defined in claim 25, wherein:
the living organism is a domestic animal.

34. A drinking water for animals, comprising:
extracts of vegetable organisms of cupressaceae served for suppressing pathogenic infections of the animals such as domestic animals, and domestic fowls.

35. The drinking water for animals as defined in claim 34, wherein:
the extracts are extracted oils of vegetable organisms of cupressaceae.

36. The drinking water for animals as defined in claim 35, wherein:
the extracted oils contain lipophilic components in the extracts obtained by steam distillation of a crushed material or powder of vegetable organisms of cupressaceae.

37. The drinking water for animals as defined in claim 34, wherein:
a pathogen of the infection to be suppressed is an influenza virus.

38. The drinking water for animals as defined in claim 37, wherein:
the pathogen of the infection to be suppressed is an avian influenza virus.

39. The drinking water for animals as defined in claim 34, wherein:
the pathogen of the infection to be suppressed is salmonella.
